(19) United States
(12) Patent Application Publication
(10) Pub. No.: US 2025/0382255 A1
(43) Pub. Date: Dec. 18, 2025

(54) ERYTHRITOL ESTER OF PHENYL BUTYRIC ACID

(71) Applicant: AAYAM THERAPEUTICS LLC, San Jose, CA (US)

(72) Inventor: Sundeep DUGAR, San Jose, CA (US)

(21) Appl. No.: 19/111,500

(22) PCT Filed: Sep. 18, 2023
(86) PCT No.: PCT/US2023/033006

§ 371 Date: Mar. 13, 2025

Related U.S. Application Data
(60) Provisional application No. 63/407,231, filed on Sep. 16, 2022.

(51) Int. Cl. C07C 69/612; A61K 31/216; C07C 67/08

(52) U.S. Cl. CPC C07C 69/612; A61K 31/216; C07C 67/08

(57) ABSTRACT

The present application discloses an erythritol analog of phenylbutyrate, and compositions including same. Also disclosed are methods of using the erythritol analog of phenylbutrate.

### FIELD OF THE INVENTION

The present invention is drawn to the field of pharmaceutical sciences. In particular, the present invention is drawn to erythritol ester of phenylbutyric acid to treat disorder associated with mutations in SLC6A1, STXBP1, SYNGAP, FOXG1 genes and other disorders or conditions associated endoplasmic reticulum stress, a process for preparation of the compound, a composition containing the compound and uses thereof.

### BACKGROUND OF THE INVENTION

Phenylbutyrate esters of alcohol are known in the art. For instance, EP2607367 discloses mono, di or triphenyl butyrate of glycerol for the treatment of cancer. U.S. Pat. No. 5,945,407 discloses tetra-butyrate ester of threitol, particularly a pharmaceutical composition comprising tetra-butyrate ester of threitol for the treatment of cancer selected from the group consisting of carcinomas, myelomas, melanomas, lymphomas and leukemia. EP0858441 discloses mono, di or tri-phenylbutyrate of glycerol for the treatment of diseases of nitrogen accumulation, patients with certain β-hemoglobinopathies, anemia, and cancer. U.S. Pat. No. 6,060,510 discloses mono, di or tri-phenylbutyrate of glycerol for the treatment of diseases of nitrogen accumulation, patients with certain β-hemoglobinopathies, anemia, and cancer. US20140206763 discloses phenylbutyrate ester of glycerol for the treatment of sporadic inclusion-body myositis and disorders relating to autophagy impairment or amyloid beta 42 accumulation. US20140155358 discloses only glyceryl tributyrate to boost the innate antimicrobial activity in the lung, trachea, urinary tract or kidney, jejunum, ileum. WO2013079205 discloses a pharmaceutical composition comprising glyceryl 4-phenylbutyrate (phenylbutyrate ester of glycerol) as the active ingredient for treating various diseases and disorders including neurodegenerative disorders. It does not disclose or suggest any other alternative for glyceryl 4-phenylbutyrate. WO2010025303 discloses phenylbutyrate ester of glycerol for the treatment of patients with nitrogen retention states, including urea cycle disorders (UCDs), cirrhosis complicated by hepatic encephalopathy (HE) and chronic renal failure.

However, none of the prior art compounds, disclose phenylbutyrate ester of erythritol. For the first time the present invention discloses phenylbutyrate esters of erythritol and demonstrates that these compounds, possess enhanced therapeutic efficacy over other compounds in the art.

### SUMMARY OF THE INVENTION

The present invention is drawn to phenylbutyrate ester of erythritol, a process for preparing the same, a composition containing the compound and uses thereof.

### DESCRIPTION OF THE INVENTION

The present invention is drawn to erythritol analogue of phenylbutyrate ester and is used to treat SLC6A1 and related disorders that include neurodevelopmental disorders including epilepsy and intellectual disability.

Solute carrier family 6 member 1 (SLC6A1)-related disorders are emerging as a common cause of developmental and epileptic encephalopathies, since initial descriptions in 2015 (Carvill et al., 2015). SLC6A1 encodes the GABA transporter protein type 1 (GAT1), which is responsible for the reuptake of GABA into presynaptic neurons and glia (Bröer and Gether, 2012). Disruption of SLC6A1 is a prominent cause of neurodevelopmental disorders, including autism spectrum disorder, intellectual disability and seizures of varying types and severity. In the current three largest genomic screens of individuals with epilepsy (8565, 9170 and 9769 patients, respectively), SLC6A1 was listed among the top 10-20 genes with the highest number of pathogenic variants (Lindy et al., 2018; Epi25 Collaborative, 2019, p. 25; Truty et al., 2019). In the most extensive autism sequencing study to date (N=11 986), SLC6A1 was among the top 10 genes, with the most significant variant enrichment in autism patients compared to 23 598 controls (Satterstrom et al., 2019). Recently, exome sequencing of individuals with schizophrenia found rare de novo missense variants in SLC6A1 to be associated with schizophrenia in three patients (Rees et al., 2020), extending the phenotype spectrum beyond epilepsy. Overall, the incidence of SLC6A1-related disorders is estimated to be 2.65 (90% CI: 2.38-2.86) per 100 000 births (López-Rivera et al., 2020).

The present invention is drawn to (2R,3S)-butane-1,2,3,4-tetrayl tetrakis(4-phenylbutanoate) and (2S,3R)-4-hydroxybutane-1,2,3-triyl tris(4-phenylbutanoate).

The compound of the present invention is provided here below at formula (I) and formula (II) (“enumerated compound(s)).

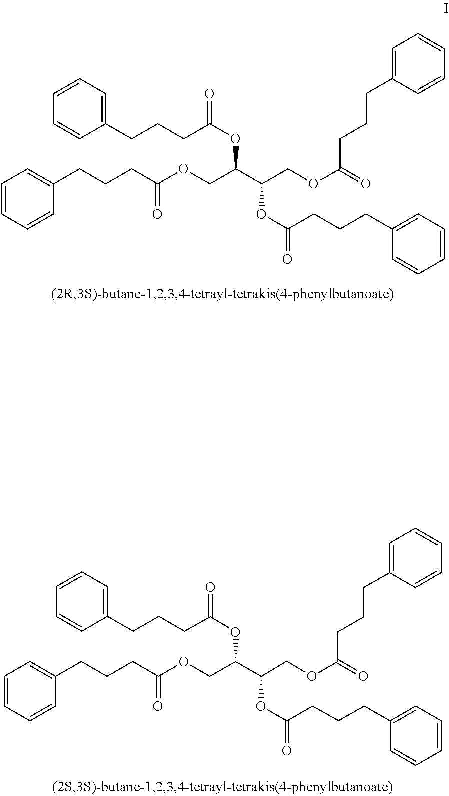 [SMILES: O=C(CCCc1ccccc1)OC[C@H](OC(=O)CCCc1ccccc1)[C@@H](COC(=O)CCCc1ccccc1)OC(=O)CCCc1ccccc1 ; O=C(CCCc1ccccc1)OC[C@H](OC(=O)CCCc1ccccc1)[C@H](COC(=O)CCCc1ccccc1)OC(=O)CCCc1ccccc1]

Without being limited by theory, it is submitted that the structural features of the compound of the present invention results in enhanced pharmacokinetics of the compounds with other prior art inventions. This is also evidenced by enhanced activity over other ester derivates of prior art as set out in the examples herein below.

In another aspect, disclosed is a composition comprising (2R,3S)-butane-1,2,3,4-tetrayl-tetrakis(4-phenylbutanoate) and/or (2S,3S)-butane-1,2,3,4-tetrayl-tetrakis(4-phenylbutanoate), and a pharmaceutically acceptable

By “pharmaceutically acceptable” is meant that the carrier, diluent or excipient must be compatible with the other ingredients of the formulation and not deleterious to the recipient thereof.

A “pharmaceutically acceptable carrier” may include pharmaceutically-acceptable material, composition or vehicle, such as a liquid or solid filler, diluent, excipient, solvent or encapsulating material, involved in carrying or transporting the disclosed compositions or active agents from one organ, or portion of the body, to another organ, or portion of the body without affecting its biological effect.

According to further embodiments, pharmaceutical formulations are provided that include compounds of the invention described herein and formulated for a specific mode of delivery. Formulations may be prepared by methods well-known in the pharmaceutical arts. For example, the enumerated compounds are brought into association with a carrier and/or diluent, as a suspension or solution. Optionally, one or more accessory ingredients (e.g., buffers, flavoring agents, surface active agents, and the like) also are added. The choice of carrier is well within the purview of the person of ordinary skill in the relevant art and is determined by the solubility and chemical nature of the compounds, chosen route of administration and standard pharmaceutical practice. These accessory ingredients and materials are well known in the art and include, but not limited to, one or more the types of accessory ingredients selected from (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents

In another embodiment, provided is a method of treating SLC6A1 and related disorders that include neurodevelopmental disorders including epilepsy and intellectual disability by administering a therapeutically effective amount of a compound or composition described herein the subject in need. The terms “treat”, “treating” or “treatment of” as used herein refers to providing any type of medical management to a subject. Treating includes, but is not limited to, administering a composition to a subject using any known method for purposes such as curing, reversing, alleviating, reducing the severity of, inhibiting the progression of, or reducing the likelihood of a disease, disorder, or condition or one or more symptoms or manifestations of a disease, disorder or condition. The term “therapeutically effective amount” relates to a dose of the substance that will lead to the desired pharmacological and/or therapeutic effect. The desired pharmacological effect is, to alleviate a condition or disease described herein, or symptoms associated therewith. A therapeutically effective amount of a substance may vary according to factors such as the disease state, age, sex, and weight of the individual, and the ability of the substance to elicit a desired response in the individual. Dosing regimen may be adjusted to provide the optimum therapeutic response. For example, several divided doses may be administered daily, or the dose may be proportionally reduced as indicated by the exigencies of the therapeutic situation.

For administration to a suitable subject, preferably to a human patient suffering from or suspected of suffering from SLC6A1 and related disorders that include neurodevelopmental disorders including epilepsy and intellectual disability, the compounds described here are prepared according to methods known in the art into suitable formulations for any route of administration and suitable doses. Suitable subjects for administration and treatment can be any mammal, including rats, mice, dogs, cats, farm animals such as cattle, sheep, horses and the like or any mammal.

The appropriate dose of an enumerated compound depends upon a number of factors within the ken of the ordinarily skilled physician, veterinarian, or researcher for example, the identity, size, and condition of the subject or sample being treated, further depending upon the route by which the composition is to be administered, the frequency of administration, the severity of the disease, and the effect which the practitioner desires the an active agent to have. Furthermore, appropriate doses of an active agent depend upon the potency with respect to the target to be modulated. Such appropriate doses may be determined using the assays described herein or which are convenient to the practitioner and know in the art. When one or more of these active agents are to be administered to an animal (e.g., a human), a relatively low dose may be prescribed at first, with the dose subsequently increased until an appropriate response is obtained. In addition, the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Dosages and regimens for administration are determined by the person of skill, including physicians. Administration of compositions, including the compounds described here, can be performed a single time, or repeated at intervals, such as by continuous infusion or repeated oral doses, over a period of time, four times daily, twice daily, daily, every other day, weekly, monthly, or any interval to be determined by the skilled artisan based on the subject involved. Treatment can involve administration over a period of one day only, a week, a month, several months, years, or over a lifetime. Regimens and duration can vary according to any system known in the art, as is known to the skilled person.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions for rectal or vaginal administration may be presented as a suppository, which maybe prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise one or more agent(s)/compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

In another aspect, the present invention provides a process for synthesis of the compound of formula (I) of the present invention. The compound of the present invention can be preferably synthesized by a process containing the steps of:
(i) Coupling erythritol with phenyl butyric acid in the presence of a coupling agent and a solvent optionally in the presence of a base to obtain the product
(ii) Optionally purifying the product of step (i) to obtain the pure stereoisomer

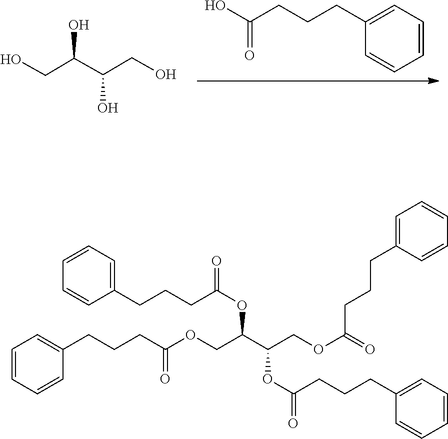 [SMILES: OC[C@@H](O)[C@@H](O)CO ; O=C(O)CCCc1ccccc1 ; O=C(CCCc1ccccc1)OC[C@H](OC(=O)CCCc1ccccc1)[C@@H](COC(=O)CCCc1ccccc1)OC(=O)CCCc1ccccc1]

The coupling agent of step (i) may be selected from reagents known to a person reasonably skilled in the art such as N,N-dicyclohexylcarodiimide or 1-Ethyl-3-(3-dimethylaminopropyl) carbodiimide or any other agent that can promote the coupling of an acid to an alcohol to make an ester.

The base can be 4-dimethylaminopyridine or other similar base known to a person reasonably skilled in the art.

The solvent of coupling in step (i) may be selected from the group consisting of dimethylformamide, dichloromethane or any other suitable solvent known to a person reasonably skilled in the art.

The coupling reaction with phenyl butyric acid at step (i) may be carried out at ambient temperatures

The purification at step (ii) may be carried out by a suitable purification method such as column chromatography or distillation under vacuum.

In another aspect, the present invention provides a process for synthesis of the compound of formula (I) of the present invention. The compound of the present invention can be preferably synthesized by a process containing the steps of:
(i) Treating a solution of erythritol with 4-phenylbutanoyl chloride in the presence of a base and a solvent to obtain the product
(ii) Optionally purifying the product of step (i) to obtain the pure stereoisomer

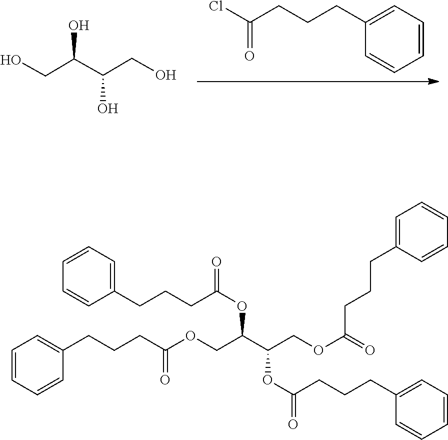 [SMILES: OC[C@@H](O)[C@@H](O)CO ; O=C(Cl)CCCc1ccccc1 ; O=C(CCCc1ccccc1)OC[C@H](OC(=O)CCCc1ccccc1)[C@@H](COC(=O)CCCc1ccccc1)OC(=O)CCCc1ccccc1]

In yet another embodiment, the present invention discloses a composition/formulation containing the compound of the present invention along with pharmaceutically acceptable excipients.

In an aspect, the present invention discloses that the use of the compound and the composition containing the compound of the present invention for its use in SLC6A1 disorders that include neurodevelopmental disorders including epilepsy and intellectual disability.

The present invention is illustrated by way of examples. The examples are meant only for illustrative purposes and should not be construed as limiting.

a. Chemistry

### Synthesis of (2R,3S)-butane-1,2,3,4-tetrayl-tetrakis(4-phenylbutanoate) (I)

### Synthetic Scheme:

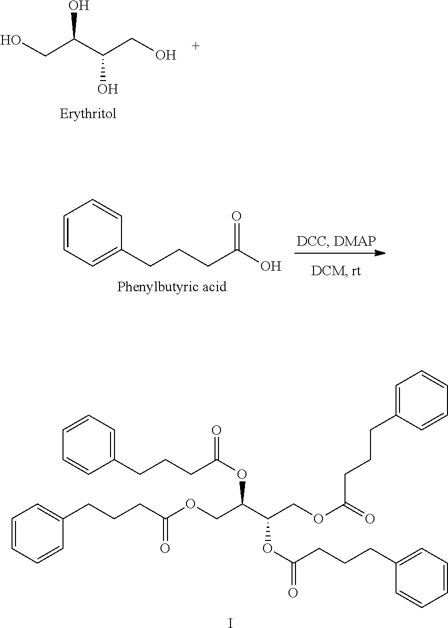

Procedure: A solution of N,N-dicyclohexylcarbodiimide (1.3 g, 6.30 mmol, 5.12 eq.) in dichloromethane (10 mL) was slowly added to a mixture of Erythritol (150 mg, 1.23 mmol, 1 eq.), 4-Phenylbutyric acid (908 mg, 5.53 mmol, 4.5 eq.), 4-Dimethylaminopyridine (75 mg, 0.61 mmol 0.5 eq.) in dichloromethane (20 ml). The reaction mixture was stirred for 14 h at room temperature (25-35° C.) under Argon atmosphere and then 1 h at 0° C. The reaction mixture was passed through celite to remove solid by product and washed with cold dichloromethane (50 mL×2). The organic solvent was dried using sodium sulphate and was evaporated in high vacuo to afford crude compound. The product obtained was purified by column chromatography (using 100-200 mesh and 1-2% Methanol-dichloromethane) to get desired compound (2R,3S)-butane-1,2,3,4-tetrayl tetrakis(4-phenylbutanoate) (Yield: 350 mg, 40%).

ESI-MS (m/z) Calcd for C₄₄H₅₀O₈: 706.87, Observed 724.4 [M+18]⁺

¹H-NMR (300 MHz, DMSO-d₆) δ: 7.25-7.22 (m, 8H), 7.18-7.13 (m, 12H), 5.24 (s, 2H), 4.34-4.31 (d, J=9 Hz, 2H), 4.20-4.16 (m, 2H), 2.56-2.54 (m, 8H), 2.31-2.24 (m, 8H), 1.82-1.75 (m, 8H)

### B. Pharmacokinetic Profile of the Compound of the Present Invention

Pharmacokinetics (PK) study was carried out to evaluate the plasma exposure of two PBA analogues (ATX-2022-018 and ATX-2022-034), when dosed orally (PO) at 22.5 mg/kg in 50% Propylene glycol. Dosing was done in overnight fasted SD rats (n=6). After oral (PO) administration blood was collected by serial bleeding at 8 different time points in heparinized tubes. Blood samples were centrifuged at 10,000 rpm for 5 min. at 4° C. to obtain the plasma, which were aspirated into separate labeled tubes and stored at −80° C. Extraction solvent was added to plasma (150 μL 0.1% acetic acid in water: 0.1% acetic acid Methanol, 20:80) was vortexed and shaken on shaker for 10 minutes, centrifuged at 10,000 rpm for 10 minutes at 4° C. Supernatant was used for LC-MS analysis. Parent drug generated that is PBA was monitored by LC-MS (API3200).

Details of the LC column and mobile phase used for the analysis is provided below:
Column: Gemini NX C18, 50 mm*4.6 mm*3.5μ
Mobile Phase: A: 0.1% Acetic in Water
   B: 0.1% Acetic acid in Methanol
Flow rate: 0.600 mL/min, gradient program

### Gradient Program:

| Time (min) | Flow rate (mL) | Pump A (%) | Pump B (%) |
|---|---|---|---|
| 0.01 | 600 | 98 | 2 |
| 1.00 | 600 | 80 | 20 |
| 1.40 | 600 | 15 | 85 |
| 1.80 | 600 | 15 | 85 |
| 1.90 | 600 | 5 | 95 |
| 3.80 | 600 | 5 | 95 |
| 4.60 | 600 | 98 | 2 |
| 6.80 | 600 | 98 | 2 |

Acetonitrile and plasma calibration curves were generated and percentage of drug recovery from plasma determined. Quantitative analysis was done by liquid chromatography tandem mass spectrometer (API3200 LC-MS/MS). Cmax, Tmax, AUC and t½ were calculated using Graph Pad PRISM version 5.04.

| | THREITOL | ERYTHRITOL | |
|---|---|---|---|
| | ANALOG OF | ANALOG OF | |
| Parameters | PBA | PBA | CHANGE |
| Vehicle | PG | PG | None |
| Dose (mg/kg) | 22.5 | 22.5 | None |
| Cmax (nM) | 12,464 | 51,299 | Increase 4-fold |
| Tmax (h) | 0.3 | 0.2 | NS |
| AUC (nM · h) | 37,594 | 104,814 | Increase 3-fold |
| MRT (h) | 0.4 | 0.6 | Increase 1.5-fold |
| Vd (ml) | 7,161 | 2,600 | Decrease 2.75-fold |
| CL (ml/h) | 600 | 225 | Decrease 2.7-fold |

What is claimed is:

1. A compound comprising the following structure:

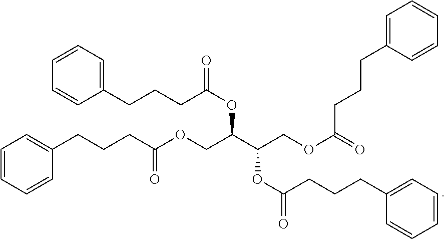 [SMILES: O=C(CCCc1ccccc1)OC[C@H](OC(=O)CCCc1ccccc1)[C@@H](COC(=O)CCCc1ccccc1)OC(=O)CCCc1ccccc1]

2. A composition comprising a compound of claim 1, and a pharmaceutically acceptable carrier.

3. The composition of claim 2, wherein the composition comprises less than 20%, 15%, 10%, 5%, 2% or 1%, by weight, of the following compound 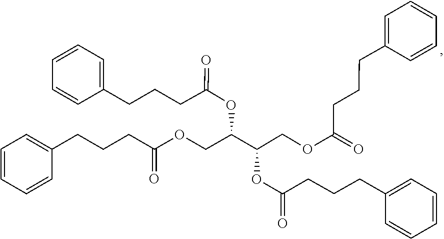 [SMILES: O=C(CCCc1ccccc1)OC[C@H](OC(=O)CCCc1ccccc1)[C@H](COC(=O)CCCc1ccccc1)OC(=O)CCCc1ccccc1]
relative to the weight percent of the compound of claim 1.

4. A method of treating a SLC6A1 and related disorders that include neurodevelopmental disorders including epilepsy and intellectual disability in a subject, the method comprising administering a therapeutically effective amount of the compound of claim 1 or composition of claim 2 or 3 to the subject.

5. A method of synthesizing the compound of claim 1, the method comprising
(i) Treating a solution of erythritol with 4-phenylbutanoyl chloride in the presence of a base and a solvent to obtain the product; and
(ii) Optionally purifying the product of step (i) to obtain a pure stereoisomer.
